(19) United States
(12) Patent Application Publication
(10) Pub. No.: US 2012/0065500 A1
(43) Pub. Date: Mar. 15, 2012

(54) RADIOPAQUE EMBEDDED INTO DESICCANT FOR IMPLANTABLE MEDICAL DEVICE

(75) Inventors: Charles R. Rogers, Maple Grove, MN (US); David D. Differding, Edina, MN (US); John M. Gray, Brooklyn Park, MN (US)

(73) Assignee: MEDTRONIC, INC., Minneapolis, MN (US)

(21) Appl. No.: 12/882,786

(22) Filed: Sep. 15, 2010

(51) Int. Cl. A61B 6/00; A61F 2/02

(52) U.S. Cl. 600/431; 623/11.11

(57) ABSTRACT

A molded desiccant article for placement in an implantable medical device includes an affixed radiopaque marker. The radiopaque marker may serve to provide information regarding the make and model of the device or may be used for purposes of determining whether the desiccant was placed in the device.

### FIELD

The present disclosure relates to implantable medical devices, particularly to implantable medical devices having a desiccant, and to molded desiccant articles.

### BACKGROUND

Implantable medical devices include a radiopaque marker for purposes of identifying the device manufacturer and model. Such device identification markers are x-ray visible and are distinguishable from other components of the device. The identification markers are valuable in emergency situations to allow a physician or other health care provider to determine whether a patient has a an implanted device, the make and model of the device, and whether any special precautions may be needed in treating the patient in the emergency situation in light of the implanted device.

The size of implantable medical devices has tended to get smaller and smaller with time, making it difficult to find an appropriate location in which to place a radiopaque identification marker. In many cases, the markers are attached to the inside of a housing of the device by an adhesive, such as a pressure sensitive adhesive. However with decreasing sizes of devices, it may not be practical to continue to adhere the radiopaque identification tag to the inside of the housing. In addition, the fixation of the identification marker to the housing can add extra materials or parts, such as double-sided pressure sensitive adhesive tape, epoxy, etc., and can add additional fixturing or process steps. In addition, such mechanisms for attaching the identification marker to the device may come loose as time wears on. For devices that include electrical or moving mechanical parts, a loose radiopaque marker within the housing can be problematic. It is also worth noting that it is often forgotten to place the marker into the device, as it is typically one of the last steps in manufacturing the device

Another component or material that is often placed in implantable medical devices having electrical components is a desiccant. The desiccant helps to prevent corrosion, short circuiting, or performance shifts of the conduction pathways. The desiccant can be molded, by combining with a suitable polymer, and placed in an available space of an implantable medical device. The device may be nearly fully assembled; the molded desiccant may then be added to occupy otherwise unused space of the device; and the final assembly of device with desiccant may be completed. However, in some cases, the desiccant may be omitted from the final assembly steps, and the detection of the lacking desiccant can be difficult.

### BRIEF SUMMARY

The present disclosure describes, among other things, a molded desiccant article for placement in an implantable medical device. A radiopaque marker is affixed (e.g., adhered, molded into, embedded in, or the like) to the molded desiccant article. The radiopaque marker may serve to provide information regarding the make and model of the device or may be used for purposes of determining whether the desiccant was placed in the device.

In various embodiments described herein, an article for placement in an implantable medical device includes a molded part formed from a polymeric material and a desiccant. The article further includes a radiopaque marker affixed to molded part.

In various embodiments described herein, an implantable medical device includes a housing, an electronic component disposed in the housing, a molded desiccant article disposed in the housing, and a radiopaque marker affixed to the molded desiccant article.

In various embodiments described herein, a method includes x-raying an implantable medical device suspected of having a desiccant article that has an affixed radiopaque marker to determine the presence of absence of the radiopaque marker. The method further includes correlating the presence or absence of the radiopaque marker with the presence or absence of the desiccant.

The devices, articles and methods described herein may provide one or more advantages over prior implantable medical devices having a desiccant or having a radiopaque marker providing information regarding the device, or methods for determining whether a desiccant was placed in the device. For example, molding or overmolding a radiopaque marker into a desiccant article simplifies the device assembly process, eliminates the need for tape or other adhesive to hold the radiopaque in place, eliminates the possibility of the radiopaque coming loose, and reduces the possibility of forgetting to put the desiccant into the device prior to final assembly. Further, the desiccant with affixed radiopaque marker allows one to verify that the desiccant was indeed placed into the device. These and other advantages of the various embodiments of the devices and methods described herein will be readily apparent to those of skill in the art upon reading the disclosure presented herein.

### BRIEF DESCRIPTION OF THE DRAWINGS

FIGS. 1-3 and 4A are schematic perspective views of embodiments of molded desiccant articles with an affixed radiopaque marker.

FIG. 4B is a schematic side view of an embodiment of the article depicted in FIG. 4A.

FIGS. 5-6 are schematic perspective views of embodiments of molded desiccant articles with an affixed radiopaque marker.

FIG. 7 is a schematic top view of an embodiment of a radiopaque marker.

FIG. 8 is a schematic view of an embodiment of a radiopaque marker.

FIG. 9 is a schematic top view of embodiments of steps in the assembly of the device.

FIG. 10 is a flow diagram of a method for assembling a device having a molded desiccant article.

FIG. 11 is a flow diagram of a method for detecting whether a device has a desiccant according to an embodiment of the teachings presented herein.

The schematic drawings presented herein are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to.” It will be understood that the terms “consisting of” and “consisting essentially of” are subsumed in the term “comprising.” For example, a microfluidic device comprising a sheet having an interconnected microporous structure, a double-sided adhesive layer, and a film may consist of, or consist essentially of, the sheet, the adhesive layer and the film.

“Consisting essentially of”, as it relates to a compositions, articles, systems, apparatuses or methods, means that the compositions, articles, systems, apparatuses or methods include only the recited components or steps of the compositions, articles, systems, apparatuses or methods and, optionally, other components or steps that do not materially affect the basic and novel properties of the compositions, articles, systems, apparatuses or methods.

Any direction referred to herein, such as “top,” “bottom,” “left,” “right,” “upper,” “lower,” “above,” below,” and other directions and orientations are described herein for clarity in reference to the figures and are not to be limiting of an actual device or system or use of the device or system. Devices or systems as described herein may be used in a number of directions and orientations.

The present disclosure describes, among other things, a molded desiccant article for placement in an implantable medical device. A radiopaque marker is affixed to the molded desiccant article. The radiopaque marker may be affixed to the molded desiccant article in any suitable manner. For example, the radiopaque marker may be incorporated into the molded desiccant article, impregnated in the article, adhered to the article, embedded in the article, molded in the article or the like. The radiopaque marker may serve to provide information regarding the make and model of the device or may be used for purposes of determining whether the desiccant was placed in the device.

The molded desiccant articles with associated radiopaque marker can be used in any suitable implantable medical device. For example, devices having a component that may corrode, short-circuit, or exhibit performance shifts in the presence of moisture may desirably include a desiccant article as described herein. Often, such devices include an electrical component. Examples of such devices include hearing implants, cochlear implants; sensing or monitoring devices; signal generators such as cardiac pacemakers or defibrillators, neurostimulators (such as spinal cord stimulators, brain or deep brain stimulators, peripheral nerve stimulators, vagal nerve stimulators, occipital nerve stimulators, subcutaneous stimulators, etc.), gastric stimulators; infusion devices; and the like.

Any one or more suitable desiccant may be used in a molded desiccant article. Examples of desiccants that may be employed include calcium oxide, silica gel, activated carbon, activated alumina, clay, other natural zeolites, anhydrous magnesium, calcium sulfate, starches, molecular sieves, aluminosilicates, and the like. In an embodiment, the desiccant comprises aluminum oxide.

The desiccant used may be molded by combining with any suitable polymeric material. Thermoplastic polymers, such as polyolefins, polyethylenes, polystyrenes and polypropylenes, may readily be used in forming a molded desiccant article. Thermoset polymers, such as silicones, styrene-butadiene polymers, and the like may also be used.

The desiccant and the polymer may be blended, mixed, or the like, prior to molding so that the desiccant is embedded in the polymer. Any suitable amount of polymer and desiccant may be used. For example, a mixture of desiccant and polymer for purposes of molding may have about 5 to about 60 weight percent desiccant and about 95 to about 40 weight percent polymer. In some embodiments, such a mixture includes about 25 to about 50 weight percent, such as between about 40 and about 50 wt %, desiccant. In some embodiments, such a mixture includes about 50 to 75 weight percent, such as between about 50 and about 60 wt %, polymer. If more than one desiccant or polymer is used, the weight percent of the polymer will be the cumulative weight percent of all of the polymers and the weight percent of the desiccant will be the cumulative weight percent of all of the desiccants.

In many cases, the mixture of desiccant and polymer will consist of, or consist essentially of, dessicant and polymer. However, in some cases one or more additives, such as a compatibilzing or coupling agent. Such agents are generally known in the art and generally make up 5% or less of the weight of the mixture.

Any radiopaque material may be used in forming a radiopaque marker. Such materials are known in the art. In some embodiments, gold, platinum, titanium or tungsten are used in forming the radiopaque marker. Of course, other materials may be used. Preferably, the marker is readily discernable from other materials of a device in which the marker is placed. For example, if the device includes components formed from titanium, it may be desirable for the marker to be formed from a radiopaque material other than titanium, such as tungsten.

The radiopaque marker may be affixed to the molded desiccant article in any suitable manner. For example, the radiopaque marker may be incorporated into the molded desiccant article, impregnated in the article, adhered to the article, embedded in the article, molded in the article or the like. In some cases, the radiopaque marker is a dye or fine material that is mixed with the polymer and desiccant prior to molding. In such cases, the radiopaque marker may not readily serve as a valuable indicia of an attribute of the device. In some embodiments, the radiopaque marker serves as an indicia of an attribute of the device, such as a capability of the device, the device manufacturer, or the device model. A code of numbers, letters, or symbols may be used to serve as the indicia. In such cases, the marker may be a sheet, plate, disc, or the like with the numbers, letters, or symbols cut or punched out. Alternatively, the marker may include one or more of the cut or punched out letters, numbers, or symbols.

When the marker serves as indicia, the marker should be of sufficient size to be detectable as the indicia, but is also preferably small enough so as to not take up much space in the device or the molded desiccant article. In various embodiments, the radiopaque marker has a thickness of 0.01 inches or less, such as about 0.008 inches. The marker may have any suitable length and width. In various embodiments, the marker has a length of 0.5 inches or less and a width of 0.5 inches or less. For example, the length may be about 0.2 inches and the width may be about 0.15 inches.

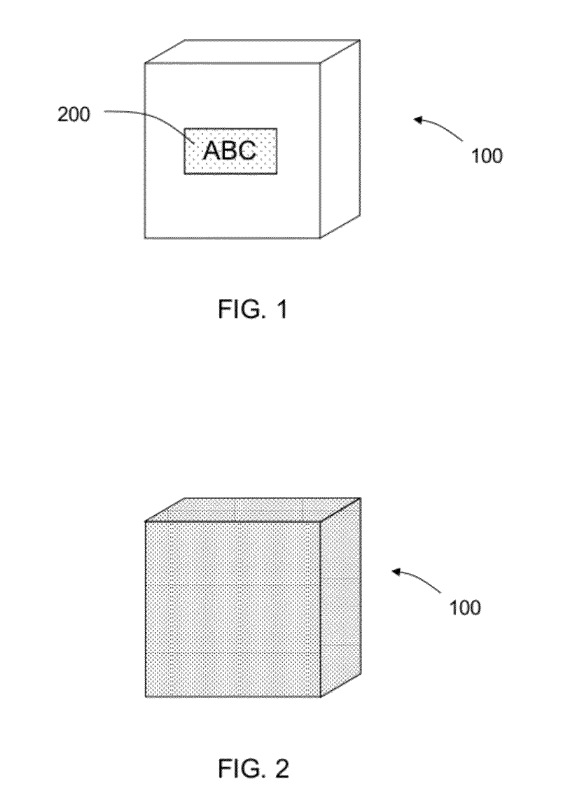

Referring now to FIG. 1, a molded desiccant article 100 having an affixed radiopaque marker 200 is shown. The marker 200 may be affixed to (which includes in) the article 100 in any suitable manner and at any suitable depth. In many cases, a molded desiccant having a desiccant and a polymer is visually opaque due to the presence of the desiccant. Thus, if the radiopaque marker 200 is positioned too far below a surface of the molded article 100, the marker 200 may not be visible. For example, see FIG. 2 in which the marker is embedded too far below the surface of the article 100 to be seen. The marker 200 may be affixed to the article 100 in either manner, i.e., so that it can be visually detected or so that it cannot be seen. In some cases, it may be desirable for the marker to be visible so that the presence of the marker in the molded desiccant article can be verified prior to placement in an implantable medical device before final assembly of the device. In some cases, it may be desirable to have the marker 200 embedded with the article 100 at a depth where it cannot be seen to ensure that the marker 100 remains affixed to (e.g. embedded in) the article 100.

While the desiccant article 100 is referred to herein as a molded article it will be understood that the desiccant article may be formed in any suitable manner, such as extrusion, provided that it includes a polymeric material and a desiccant.

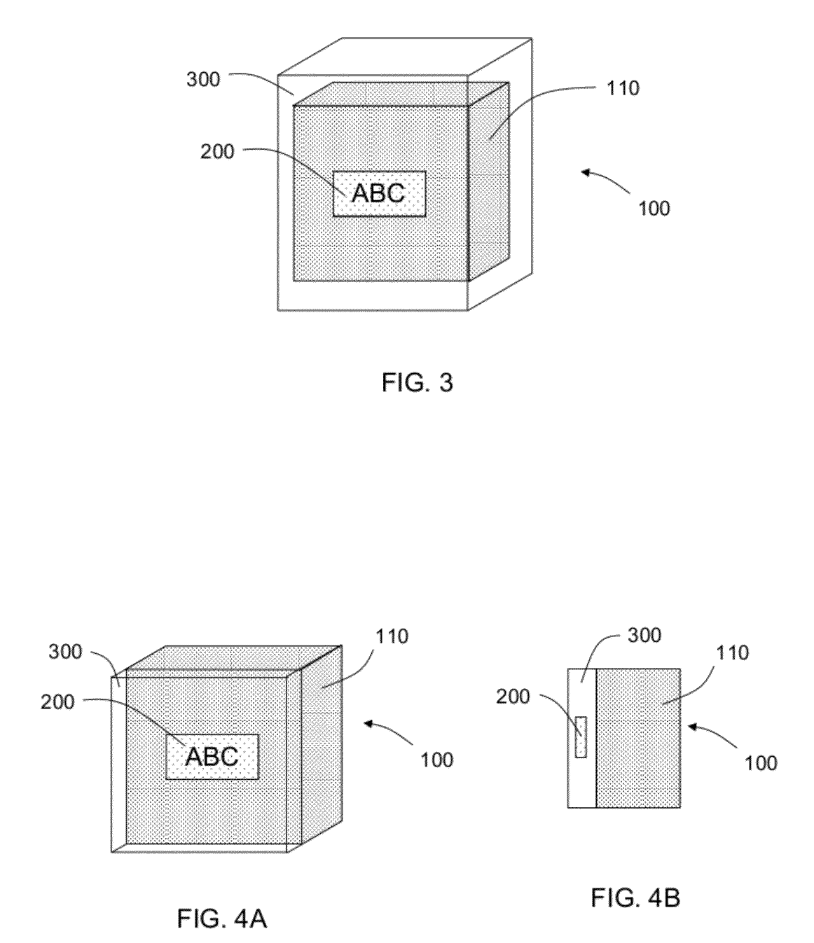

Referring now to FIGS. 3-5, embodiments of a molded desiccant article 200 having an affixed radiopaque marker 200 and an overmolded optically transparent polymer 300 are shown. As used herein, “optically transparent” means that an object lying beyond a body can be clearly seen through the body. In the depicted embodiments, the molded part 110 that includes a desiccant and a polymer is optically opaque, and the overmolded polymer 300 is optically transparent, allowing visualization of the radiopaque marker 200. The marker is at least partially embedded in the overmolded polymer 300. With reference to FIG. 3, the overmolded polymer 300 is molded about the entire surface of molded part 110. In FIGS. 4A-B, the overmolded polymer 300 is molded over one face of molded part 110. As shown in FIG. 4B, which is a side view of an embodiment of the article 100 depicted in FIG. 4A, the radiopaque marker 200 is embedded in the overmolded polymer an not molded part 100. This allows the radiopaque marker 200 to be visually observed through the optically transparent polymer overmold 300. The overmold 300 not only serves as a window for viewing the marker 200 but also serves to affix the marker 200 to the article 100. While not shown, it will be understood that the marker 200 may be partially embedded, or fully embedded at a shallow depth, in the molded part and may still be visible. The overmolded polymer 200 can serve to aid in the retention of the marker 200 in the article 100. It will be understood that the overmold 300 should not substantially interfere with the ability of the desiccant to sequester moisture. If the overmold 300 is formed from material or has a property (e.g., thickness) that may interfere with the desiccant; the amount of surface area of the article 100 that the overmold 300 covers may be limited to prevent substantial interference with the desiccant; the thickness of the desiccant may be minimized to prevent substantial interference with the desiccant; or the like.

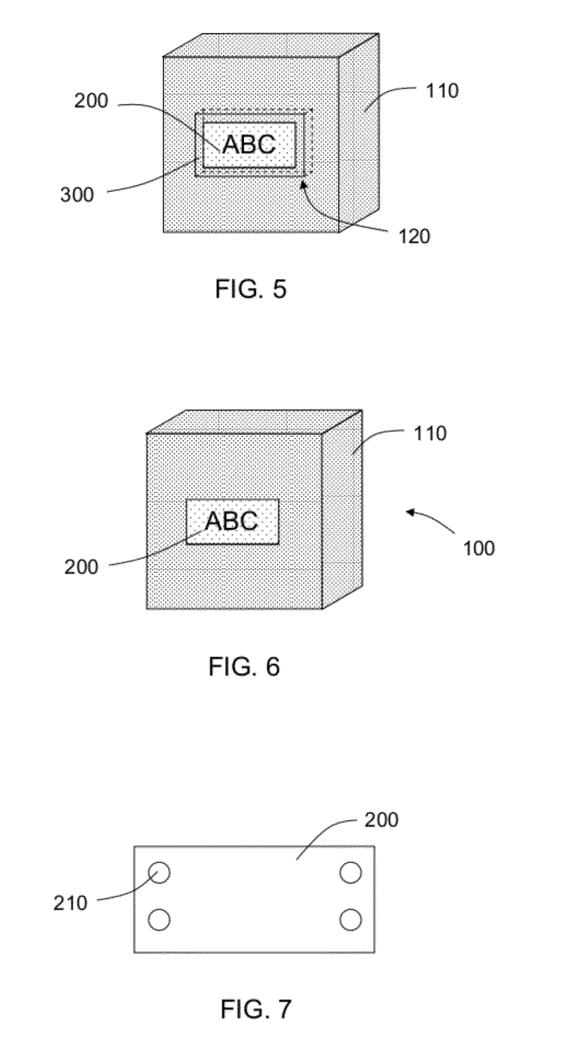

Referring now to FIG. 5, the molded part 110 has a recess 120 into which the marker 200 fits. The overmolded optically transparent polymer 300 fills the recess 120 and aids in the retention of the marker 200.

Any suitable optically transparent polymer may be used as the overmold 300 depicted in FIG. 2-5. In many embodiments, the polymer is the same as the polymer employed in the molded part that includes the desiccant. The absence of the desiccant may render the polymer optically transparent so that a marker 300 under or in the polymer can bee seen.

In some embodiments, a two-shot injection molding process may be employed to produce an article 100 as depicted in FIGS. 3-5. However, any other suitable process may be employed to produce an article 100 with an overmold 300 as depicted in FIGS. 2-5.

It will be understood that the embodiments depicted in FIGS. 3-5 are only some of the contemplated ways for affixing the marker 200 to the device 100 in a manner such that the marker 200 is visible and that any other suitable mechanism to may be used so that the marker 200 is visible.

For example, and with reference to FIG. 6, the marker 200 may be partially embedded, embedded just under the surface, or adhered to the molded part 110 so that the marker 200 is visible. In many cases, it is desirable to omit added components or process steps. Thus, in some cases, it may be desirable to affix the marker 200 to the device 100 without the use of an adhesive, such as a pressure sensitive adhesive, and epoxy, of the like. Thus, it may be desirable to at least partially embed the marker 200 in the molded part 110 to affix the marker 200 to the device 100. Any suitable process may be employed to partially embed the marker 200 in, or embed the marker 200 just under the surface of, the molded part 110. For example, the marker 200 may be placed at a surface of a mold prior to filling the mold with the mixture including the desiccant and the polymer. By placing the marker at a surface of the mold, at least the portion of the marker that contacts the surface of the mold will not be embedded in the resulting molded part.

Referring now to FIG. 7, the marker 200 may include one or more retention holes 210 to facilitate retaining the marker 200 on a molded device. The holes are sized to allow material being molded to pass through the holes and retain the marker to the molded article. In some embodiments, one or more of the holes 210 are the indicia (e.g., numbers, letters or symbols) that are punched out of a radiopaque substrate.

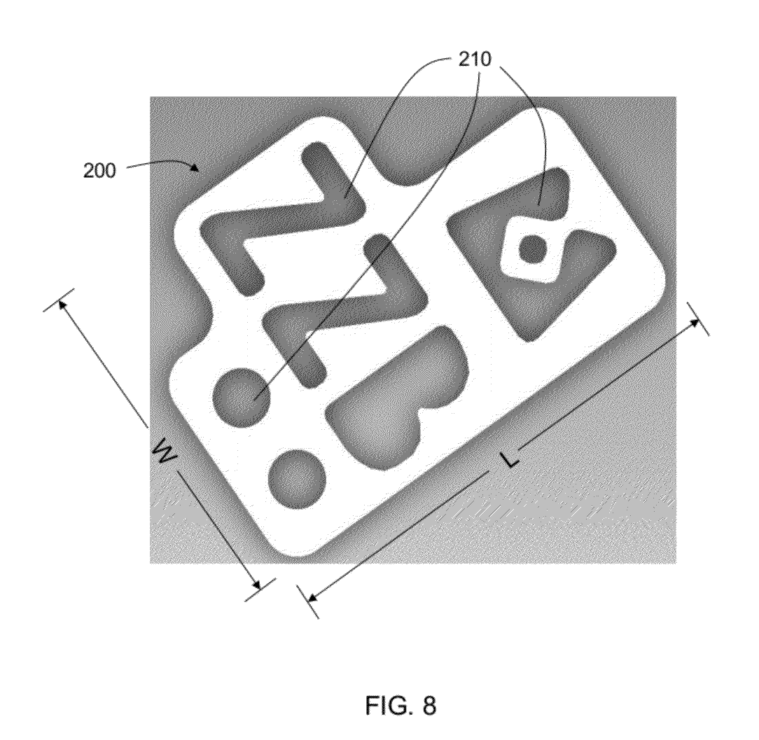

FIG. 8 is schematic drawing of an example of a radiopaque marker 200 having indicia (shown as “ZZB”) regarding an attribute of a device. The marker includes retention holes, and the indicia also serve as retention holes. In the depicted embodiment, the marker 200 has a length L of about 0.206 inches, a width W of about 0.15 inches, and a depth of about 0.008 inches. Of course, a marker 200 may have any suitable length, width and depth and may contain any suitable indicia.

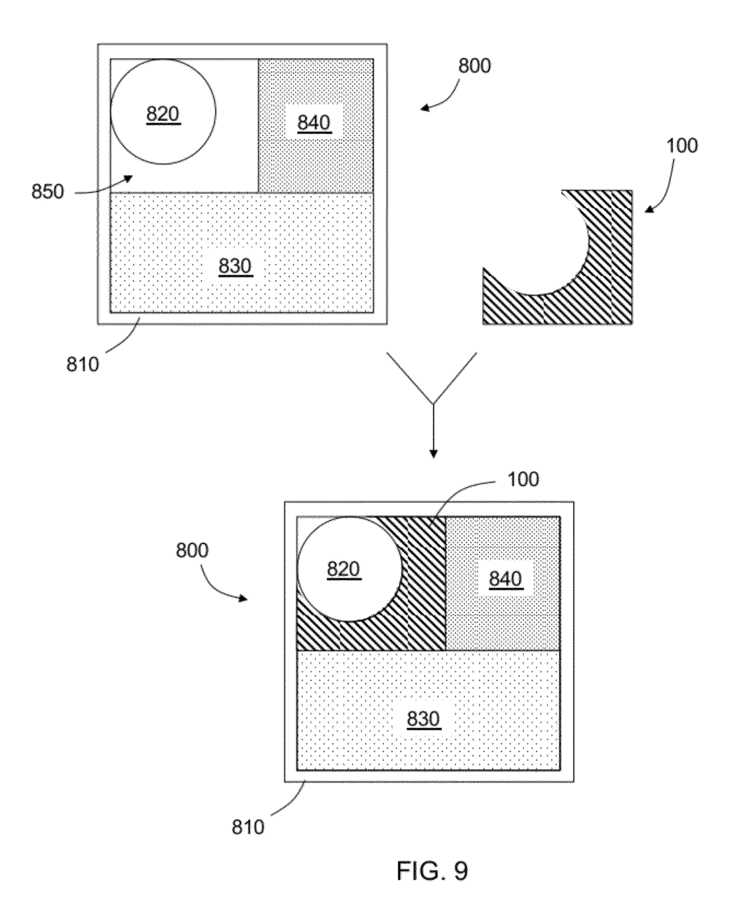

Referring now to FIG. 9, a molded desiccant article 100 having a radiopaque marker (not shown) may be placed into an implantable medical device 800. The molded article 100 is preferably formed to fill a free space 850 in the device. Depending on the design, assembly, and components of the device 800, the desiccant article 100 may be molded to occupy any suitable free space 850. Thus, the desiccant article 100 with affixed radiopaque marker can be made to fit in the device 800 without adding substantial volume to the device 800 when fully assembled.

During assembly of the device 800, one or more components 820, 830, 840 are placed within a housing 810 or a partial housing of the device. The molded desiccant article 100 may then be placed in the housing 810 to occupy free space 850. The housing 810 may then be sealed to complete final assembly of the device 800. In many embodiments, at least one of the components 820, 830, 840 of the device 800 is an electronic component. The device 800 may include any electronic component, such as a microprocessor, volatile or non-volatile memory, a switch, a circuit board, a power supply, a resistor, or the like.

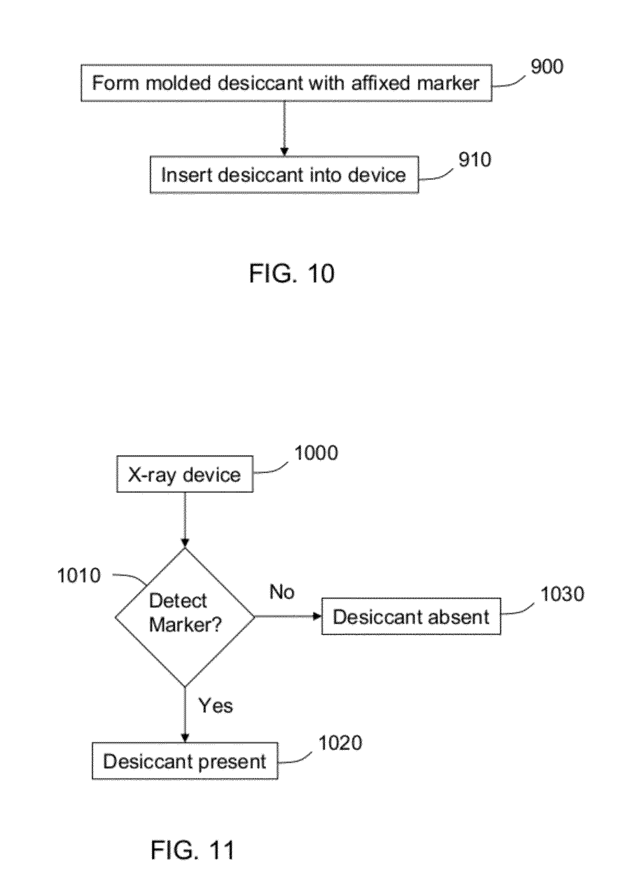

Referring now to FIG. 10 an overview of method is shown. The method includes forming a molded desiccant having an affixed radiopaque marker (900) and inserting the molded desiccant article into the device (910). The molded desiccant article can be made in any suitable manner, such as described above, and the radiopaque marker can be affixed to the article in any suitable manner, such as described above. The article may be inserted into the device at any suitable point in the assembly of the device. Typically, the molded desiccant article with affixed radiopaque marker will be inserted near the end of the assembly process, just prior to sealing the housing.

Referring now to FIG. 11, a method for determining whether an implantable medical device contains a desiccant article is shown. The method can serve as a valuable check for manufacturer prior to shipping the device. The device may be one that is supposed to have a molded desiccant article with an affixed radiopaque marker. The method includes x-raying the device suspected of a desiccant article with an affixed radiopaque (1000) and determining whether the x-ray indicates the presence of the radiopaque marker (1010). If the radiopaque marker is present, a determination may be made that the desiccant is present (1020). If the radiopaque marker is not present, a determination may be made that the desiccant is not present (1030).

Thus, embodiments of RADIOPAQUE EMBEDDED INTO DESICCANT FOR IMPLANTABLE MEDICAL DEVICE are disclosed. One skilled in the art will appreciate that the apparatuses and methods described herein can be practiced with embodiments other than those disclosed. The disclosed embodiments are presented for purposes of illustration and not limitation.

What is claimed is:

1. An article for placement in an implantable medical device, comprising:
a part comprising a polymeric material and a desiccant; and
a radiopaque marker affixed to part.

2. The article of claim 1, wherein the article is shaped to occupy a free space in the implantable medical device during final assembly of the device.

3. The article of claim 1, wherein the radiopaque marker is an indicia of an attribute of the device.

4. The article of claim 1, wherein the radiopaque marker comprises tungsten.

5. The article of claim 1, wherein the polymeric material comprises silicone.

6. The article of claim 5, wherein the desiccant comprises aluminum oxide.

7. The article of claim 1, wherein the article further comprising an optically transparent polymer molded over at least a portion of the part, wherein the radiopaque marker is at least partially embedded in the overmolded optically transparent polymer.

8. The article of claim 7, wherein the polymeric material of the molded part and the optically transparent overmolded polymer are the same.

9. The article of claim 1, wherein the radiopaque marker is at least partially embedded in the part.

10. An implantable medical device, comprising:
a housing;
an electronic component disposed in the housing;
a desiccant article disposed in the housing;
a radiopaque marker affixed to the molded desiccant article.

11. The implantable medical device of claim 10, wherein the desiccant article comprises a molded part that includes a polymeric material and a desiccant.

12. The implantable medical device of claim 11, wherein the polymeric material comprises silicone and the desiccant comprises aluminum oxide.

13. The implantable medical device of claim 11, wherein the desiccant article further comprises an optically transparent polymer molded over at least a portion of the molded part, and wherein the radiopaque marker is at least partially embedded in the overmolded optically transparent polymer.

14. The implantable medical device of claim 11, wherein the radiopaque marker is at least partially embedded in the molded part.

15. The implantable medical device of claim 10, wherein the radiopaque marker is an indicia of an attribute of the device.

16. The implantable medical device of claim 10, wherein the device is selected from the group consisting of an infusion device, an electrical signal generator, and a monitoring device.

17. The implantable medical device of claim 10, wherein the radiopaque marker is x-ray visible and distinguishable from other components of the device in an x-ray.

18. The implantable medical device of claim 10, wherein the radiopaque marker comprises tungsten.

19. A method comprising:
x-raying an implantable medical device suspected of having a desiccant article, wherein a radiopaque marker is affixed to the desiccant article, to determine the presence of absence of the radiopaque marker; and
correlating the presence or absence of the radiopaque marker with the presence or absence of the desiccant.

20. A method for manufacturing a medical device such that the presence of a desiccant can be detected, comprising:
placing a desiccant article into the device prior to final assembly of the device, wherein a radiopaque marker is affixed to the desiccant article.
